(19) United States
(12) Patent Application Publication
(10) Pub. No.: US 2022/0040100 A1
(43) Pub. Date: Feb. 10, 2022

(54) Stable Acyclovir Sodium Parenteral Formulation

(71) Applicant: RK Pharma Solutions LLC, Piscataway, NJ (US)

(72) Inventors: Ravishanker Kovi, Monroe Township, NJ (US); Raghu Kasu, Monmouth Jct, NJ (US); Thupalli Ajeykumar Reddy, Bangalore (IN); Vamshi Yekkanti, Telangana (IN)

(21) Appl. No.: 17/354,373

(22) Filed: Jun. 22, 2021

Related U.S. Application Data
(60) Provisional application No. 63/043,865, filed on Jun. 25, 2020.

(51) Int. Cl. A61K 9/08; A61K 31/522; A61K 47/02; A61K 9/00

(52) U.S. Cl. CPC A61K 9/08; A61K 9/0019; A61K 47/02; A61K 31/522

(57) ABSTRACT

The present application provides method for formulating and corresponding liquid parenteral formulations that include a desired amount of acyclovir sodium added to a vessel with a pharmaceutically acceptable infusion diluent, which are stirred to provide a shelf stable injectable solution.

### FIELD OF THE INVENTION

The present application relates to the preparation of parenteral formulations of Acyclovir sodium. In particular, present application relates to the preparation of Acyclovir sodium formulation in IV bags. Present application also relates to the use of this formulation in the treatment of viral infections of the skin and mucous membranes.

### BACKGROUND OF THE INVENTION

Acyclovir is a synthetic nucleoside analog active against herpesviruses. Acyclovir sodium is a white, crystalline powder with the molecular formula C₈H₁₀N₅NaO₃ and a molecular weight of 247.19. The maximum solubility in water at 25° C. exceeds 100 mg/mL. The chemical name of acyclovir sodium is 2-amino-1,9-dihydro-9-[(2-hydroxyethoxy)methyl]-6H-purin-6-one monosodium salt; it has the following structural formula:

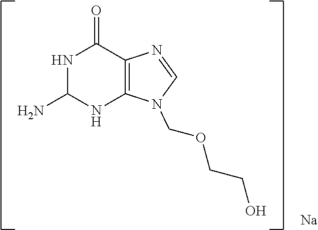

Acyclovir sodium was initially approved as lyophilised powder. But due to complexity associated with lyophilised powder, ready to use solution is developed. In sterile lyophilized powder for intravenous administration, each vial contains 500 mg of acyclovir and 49 mg of sodium, and each vial contains 1,000 mg acyclovir and 98 mg of sodium. Reconstitution of the 500-mg or 1,000-mg vials with 10 mL or 20 mL, respectively, of Sterile Water for Injection, USP results in a solution containing 50 mg/mL of acyclovir. The pH of the reconstituted solution is approximately 11(10.5-11.5).

Acyclovir sodium is an antiviral which particularly inhibits human herpes viruses, including herpes simplex types 1 (HSV-1) and 2 (HSV-2), varicella zoster, Epstein-Barr virus (EBV) and cytomegalovirus (CMV). The inhibitory activity of acyclovir is highly selective for these viruses.

Synthesis of acyclovir is discussed in U.S. Pat. No. 4,199,574, which is incorporated herein by reference. Various other publications also discusses the synthesis of acyclovir. The chemical name of acyclovir is 2-amino-1,9-dihydro-9-[(2-hydroxyethoxy)methyl]6H-purin-6-one.

Acyclovir sodium is available in injection dosage form. A number of formulations approved for Acyclovir sodium are either in lyophilised powder form or ready to use solution in vial. When such formulations are prepared, multiple impurities formulation are observed. Therefore, there is a need for a stable, susceptible sterile drug solution contained in flexible infusion bags that precludes or reduces the impurities and degradation of the API during stability. Thus, there is need to develop an Acyclovir sodium formulation in IV bags. Although, previous attempts have been made to improve the delivery and bioavailability of Acyclovir, these attempts have had limited success. Therefore, this is also one of the reasons for a development need for parenteral acyclovir formulations in IV bags.

### SUMMARY OF THE INVENTION

One aspect of the present application provides parental formulation of Acyclovir sodium.

In another aspect of the application, there is provided a storage stable injectable liquid parental composition comprising Acyclovir sodium and other pharmaceutically acceptable adjuvants.

In another general aspect of the application, the storage stable injectable compositions of the present application are useful as an anti-viral drug.

The details of one or more embodiments of the invention are set forth in the description below. Other features, objects and advantages of the application will be apparent from the description.

### DETAILED DESCRIPTION OF THE INVENTION

In order to overcome one or more of the drawbacks of the state of the art, the present application provides a ready for use injectable stable Acyclovir sodium pharmaceutical composition, which can be stored in a plastic bag and prevents the development of unwanted impurity in the solution over time.

In the context of this application “Acyclovir” refers to the pharmaceutically acceptable salts, solvates, hydrates and anhydrous forms thereof, preferably Acyclovir.

In one embodiment, liquid parenteral formulations of Acyclovir is provided that includes;
a) Acyclovir sodium,
b)—Pharmaceutically acceptable infusion diluents,
c) optionally, any other pharmaceutically acceptable adjuvants thereof, and
d) packaging containing the formulated product

Pharmaceutically acceptable infusion diluents include, but not limited to, 0.9% sodium chloride, lactated Ringer's solution and mixtures thereof.

The formulation of the present application has excellent stability at room temperature (long term) and 40° C. accelerated. The formulation according to at least one embodiment is stored in a plastic container, in particular an infusion bag. The pharmaceutical composition according to that least one embodiment is suitable for use in an infusion bag. In particular embodiment, infusion bag made of polypropylene (PP) tri-laminate, polyethylene, polyvinyl chloride or combinations of extruded polymers.

The stable, ready to use injectable solution in a bag comprising Acyclovir sodium may have concentration of Acyclovir less than 100 mg/ml, for example, to less than about 5 mg/ml. Preferably, the concentration is about 50 mg/nil and more preferably to 7 mg/ml.

The pharmaceutical acceptable adjuvants for the composition according to at least one embodiment may include one or more of the pharmaceutically acceptable solvents, co-solvents, solubilizing agents, preservatives, polymers, pH adjusting agents, chelating agents and antioxidants.

Suitable pharmaceutically acceptable solvents include, but are not limited to, ethanol, propylene glycol, glycerine, polyethylene alcohol, propylene glycol esters, polyethylene glycols, benzyl alcohol and the like. Preferred solvents are ethanol, polyethylene glycols (PEG) and propylene glycol.

Examples of pharmaceutically acceptable pH adjusting agents may include, but are not limited to, sodium hydroxide, hydrochloric acid, ammonium hydroxide, potassium hydroxide, acetic acid, calcium carbonate, magnesium carbonate, sodium carbonate, sodium bicarbonate, triethanolamine, or any combination thereof.

As used herein, and unless otherwise specified the term “about” means an acceptable error for a particular value as determined by one of ordinary skill in the art, which depends in part on how the value is measure or determined. In certain embodiments, the term about means within 10%, 9%, 8%, 7%, 6%, 5%, 4%, 3%, 2%, 1%, 0.5%, 0.1%, or 0.05% of a given value or range.

As used herein, and unless otherwise specified, the term “storage stable” refers to any composition comprising Acyclovir having sufficient physical and chemical stability to allow storage at a convenient temperature, such as between about 0° C. and about 50° C., for commercially reasonable period of time. The term “physical stability” refers to maintenance of colour or colourless state, dissolved oxygen level, head space oxygen level and particulate matter and the term chemical stability relates to formation of drug related impurities in terms of total impurities, single maximum individual impurity maximum individual unknown impurity. For pharmaceutical products, stability is required for commercially relevant times after manufacturing, such as for about 6, 12, 18, 24, or 36 months, during which a product is kept in its original packaging under specified storage condition.

In one embodiment, there is provided a storage stable injectable solution comprising Acyclovir having controlled impurity profile suitable for regulatory approval at various storage conditions.

The formulation prepared as disclosed in the present application may retain at least 90% of the potency of Acyclovir in the injectable solution after storage for 24 months at 25° C. temperature and at 60% relative humidity, and more preferably at least 99%, and yet more preferably at least 99.9% after (long) storage of about 3 months.

In one embodiment, the storage stable injectable composition according to at least one embodiment is formulated to provide single dosage administration. In another embodiment, the single dosage composition may be packed in an, Infusion bag. Infusion bag volume may vary from 60 ml to 150 ml.

In another embodiment, a storage stable injectable solution is provided that includes acyclovir having a pH value from about 7 to about 12, for example, from about 8 to about 11.5 or about 12.

In another embodiment, the storage stable ready-to-use, injectable solution comprising Acyclovir sodium has an assay of Acyclovir from about 80% to about 120%, for example, from about 90% to about 110%, or about 100%.

The present application also provides method(s) of treatment of various types of virus, such as herpes viruses, by injecting a solution as disclosed herein comprising Acyclovir in a therapeutically effective amount. Other uses include, but are not limited to, mucocutaneous, ocular, and systemic herpes simplex infections (HSV), including in human immunodeficiency virus (HIV)-infected individuals. It is also useful to treat HSV encephalitis, neonatal HSV infections, and genital herpes (first episode, recurrent and suppressive therapy for recurrent infections). Further, acyclovir as disclosed herein may be an effective therapy for varicella-zoster infections, herpes zoster (shingles, zoster), cytomegalovirus infections, infections and disorders associated with Epstein-Barr virus.

Therapeutically effective amounts of an acyclovir for use in treatment of all conditions and disorders described herein, is an amount sufficient to suppress or alleviate conditions associated with the viral infection. As will be recognized by those in the field, an effective amount of therapeutic agent will vary with many factors including the potency of the acyclovir or salt, ester, or prodrug thereof, the age and weight of the patient, and the severity of the condition or disorder to be treated.

The following examples are for the illustration of the invention only and are not intended in any way to limit the scope of the present application.

### Example 1

### Manufacturing Formula:

| Sr.no | Ingredients | Quantity |
|---|---|---|
| 1 | Acyclovir sodium | 700 mg |
| 2 | 0.9% NaCl | q.s. 100 mL |

### Manufacturing Procedure:

Step 1: 700 mg of acyclovir sodium was weighed and transferred to a manufacturing vessel.

Step 2: 0.9% NaCl solution (90 ml) was added to the acyclovir sodium in the above step and the mixture was stirred till it became clear.

Step 3: the pH of the solution was checked and adjusted as necessary to between 8.5 to 10.

Step 4: total volume was increased to 100 ml by adding 0.9% NaCl solution.

Step 5: the clear solution was used to fill an infusion bag made of polypropylene or polyvinyl chloride or LDPE bags or combination of polymer extrudes.

Step 6: The formulation was then stored at 25° C.±2° C./60% RH±5% RH and at 40° C.±2° C./75% RH±5% RH.

The Acyclovir formulation was then tested for stability at long term (25° C.±2° C./60% RH±5% RH) and at accelerated condition (40° C.±2° C./75% RH±5% RH) for 3 Months. The following stability data was observed.

### Stability Data:

| | Days | | | | | |
|---|---|---|---|---|---|---|
| | 1 Month | | 2 Months | | 3 Months | |
| Storage condition | Long term | Accelerated | Long term | Accelerated | Long term | Accelerated |
| %Purity | 99.99 | 99.99 | 99.98 | 99.93 | 99.95 | 99.89 |
| 0.85 RRT | ND | ND | ND | 0.01 | ND | 0.02 |
| Impurity-O/Q | ND | ND | 0.01 | 0.03 | 0.01 | 0.04 |
| Impurity-I | 0.01 | 0.01 | 0.02 | 0.02 | 0.02 | 0.02 |
| Impurity K | ND | ND | ND | ND | 0.01 | 0.03 |
| Total IMP | 0.01 | 0.01 | 0.03 | 0.06 | 0.04 | 0.11 |

### Example 2

### Manufacturing Formula:

| Sr.no | Ingredients | Quantity |
|---|---|---|
| 1 | Acyclovir sodium | 700 mg |
| 2 | Lactated ringer solution | q.s. 100 mL |

### Manufacturing Procedure:

Step 1: 700 mg of acyclovir sodium was weighed and transferred to a manufacturing vessel.

Step 2: a lactated Ringer's solution (90 ml) was added to the acyclovir sodium in the above step and the mixture was stirred until it became clear.

Step 3: the pH of the solution was checked and adjusted as necessary to between 8.5-10.0.

Step 4: total volume was increased to 100 ml by adding a lactated Ringer's solution.

Step 4: the clear solution was used to fill an infusion bag made of polypropylene, polyvinyl chloride or LDPE bags or combination of polymer extrudes.

Step 5: the formulation was then stored at 25° C.±20 C/60% RH±5% RH and at 40° C.±20 C/75% RH±5% RH.

This Acyclovir formulation was tested for stability at long term (25° C.±2° C./60% RH±5% RH) and at accelerated condition (40° C.±2° C./75% RH±5% RH) for 3 Months. The following stability data was observed.

| | Days | | | | | |
|---|---|---|---|---|---|---|
| | 1 Month | | 2 Months | | 3 Months | |
| Storage condition | Long term | Accelerated | Long term | Accelerated | Long term | Accelerated |
| %Purity | 99.99 | 99.99 | 99.98 | 99.94 | 99.97 | 99.92 |
| Unknown IMP - 1 | ND | ND | ND | 0.01 | ND | 0.02 |
| Impurity-O/Q | ND | ND | 0.01 | 0.03 | 0.01 | 0.02 |
| Impurity-I | 0.01 | 0.01 | 0.01 | 0.01 | 0.01 | 0.01 |
| Impurity K | ND | ND | ND | ND | 0.01 | 0.03 |
| Total IMP | 0.01 | 0.01 | 0.01 | 0.05 | 0.03 | 0.08 |

While the foregoing invention has been described in some detail for purposes of clarity and understanding, it will be appreciated by one skilled in the art, from a reading of the disclosure, that various changes in form and detail can be made without departing from the true scope of the invention.

What is claimed is:

1. A method for formulating a liquid parenteral formulation, comprising:
transferring a desired amount of acyclovir sodium to a vessel;
adding to the vessel a pharmaceutically acceptable infusion diluent;
stirring the mixture; and
transferring the acyclovir sodium solution to at least one intravenous bag.

2. The method of claim 1, wherein the pharmaceutically acceptable infusion diluent comprises a NaCl solution.

3. The method of claim 1, wherein transferring the acyclovir sodium solution comprises transferring about 60 ml to about 150 ml of the acyclovir sodium solution to the at least one intravenous bag.

4. The method of claim 1, wherein the acyclovir sodium solution has a purity of at least 99.9% after about three months of storage.

5. The method of claim 1, comprising adding a pharmaceutical acceptable adjuvant comprising at least one of a solvent, co-solvent, solubilizing agent, preservative, polymer, pH adjusting agent, chelating agent, and an antioxidant.

6. The method of claim 1, comprising adding a pharmaceutical acceptable solvent comprising at least one of ethanol, propylene glycol, glycerin, polyethylene alcohol, propylene glycol esters, polyethylene glycols, benzyl alcohol and the like.

7. A parenteral liquid formulation, comprising:
a desired amount of acyclovir sodium; and
a pharmaceutically acceptable infusion diluent, wherein the acyclovir sodium solution is stored in at least one intravenous bag.

8. The parenteral liquid formulation of claim 7, wherein the pharmaceutically acceptable infusion diluent comprises a NaCl solution.

9. The parenteral liquid formulation of claim 7, wherein a ratio of the acyclovir sodium to the infusion diluent is less than about 100 mg/ml.

10. The parenteral liquid formulation of claim 7, a pH of the stirred solution is between 8.5 to 12.

11. The parenteral liquid formulation of claim 7, wherein the acyclovir sodium solution has a purity of at least 99.9% after about three months of storage.
